(19) United States
(12) Patent Application Publication
(10) Pub. No.: US 2026/0315428 A1
(43) Pub. Date: Oct. 15, 2026

(54) INHIBITORS OF THE CERAMIDE METABOLIC PATHWAY FOR OVERCOMING IMMUNOTHERAPY RESISTANCE IN CANCER

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITÉ TOULOUSE III - PAUL SABATIER, Toulouse (FR); INSTITUT CLAUDIUS REGAUD, Toulouse (FR)

(72) Inventors: Bruno SEGUI, Toulouse Cedex 1 (FR); Nicolas MEYER, Toulouse (FR); Thierry LEVADE, Toulouse (FR); Anne MONTFORT, Toulouse cedex 1 (FR); Nathalie ANDRIEU-ABADIE, Toulouse (FR); Carine DUFAU, Toulouse cedex 1 (FR); Matthieu GENAIS, Toulouse cedex 1 (FR); Benjamin JUNG, Toulouse Cedex 1 (FR); Jean-Pierre DELORD, Toulouse (FR)

(21) Appl. No.: 19/105,469

(22) PCT Filed: Sep. 5, 2023
(86) PCT No.: PCT/EP2023/074343

§ 371 Date: Feb. 21, 2025

(30) Foreign Application Priority Data
Sep. 6, 2022 (EP) 22306318.1

(51) Int. Cl. A61K 31/225; A61K 39/00; A61K 39/395; A61P 35/00; G01N 33/5751; G01N 33/92

(52) U.S. Cl. CPC A61K 31/225; A61K 39/3955; A61P 35/00; G01N 33/5751; G01N 33/92; A61K 2039/507; G01N 2405/08

(57) ABSTRACT

Advanced cutaneous melanoma can be treated by immunotherapy targeting immune check points such as PD-1 and CTL-A4. However, 50% of patients do no respond because of primary or acquired resistance mechanism. Thus, new targets for addressing the treatment of said resistance are highly needed. The inventors show that TNF (Tumour Necrosis Factor) and ceramide metabolism alterations in melanoma cells contribute to melanoma progression and resistance to immunotherapies. In particular, the inventors demonstrate that TNF is a potent modulator of ceramide metabolism and TNF-mediated ceramide metabolism changes contribute to various biological processes such as cell proliferation, cell death and cell differentiation. Among the biological processes by which TNF triggers melanoma immune escape and resistance to immunotherapies, TNF triggers a dedifferentiation process of melanoma cells associated with the reduction of melanocytic antigen expression and epithelial to mesenchy mal transition. Finally, the inventors show that glycosphingolipid pattern in plasma can predict the clinical outcome of advanced melanoma treated with ipilimumab and nivolumab. Accordingly, ceramide metabolites and metabolizing-enzymes can be new therapeutic targets and/or biomarkers in advanced melanoma patients treated with immunotherapies.

### FIELD OF THE INVENTION

The present invention is in the field of medicine, in particular oncology.

### BACKGROUND OF THE INVENTION

Advanced cutaneous melanoma can be treated by immunotherapy targeting immune check points such as PD-1 and CTLA-4. However, 50% of patients do not respond because of primary or acquired resistance mechanism. Thus, new targets for addressing the treatment of said resistance are highly needed.

### SUMMARY OF THE INVENTION

The present invention is defined by the claims. In particular, the present invention relates to use of inhibitors of the ceramide metabolic pathway for overcoming immunotherapy resistance in cancer.

### DETAILED DESCRIPTION OF THE INVENTION

The present invention relates to a method of overcoming immunotherapy resistance in patient suffering from cancer thereof comprising administering to the patent a therapeutically effective amount of an agent that reduces or prevents the increase in ceramide and glycosylated ceramide levels.

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In some embodiments, the subject suffers from a cancer selected from the group consisting of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant, Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplasia, Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, non-small cell lung cancer (NSCLC) which coexists with chronic obstructive pulmonary disease (COPD), Ocular oncology, Oligoastrocytoma, Oligodendroglioma, Oncocytoma, Optic nerve sheath, meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastema, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute, lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Vemer Morrison syndrome, Verrucous carcinoma, Visual Pathway Glioma, Vulvar Cancer, Waldenstrom's macroglobulinemia, Warthin's tumor, Wilms' tumor, or any combination thereof. In some embodiments, the cancer is not a breast cancer.

In some embodiments, the patient suffers from melanoma.

As used herein, the term “melanoma” refers to a condition characterized by the growth of a tumor arising from the melanocytic system of the skin and other organs. Most melanocytes occur in the skin, but are also found in the meninges, digestive tract, lymph nodes and eyes. When melanoma occurs in the skin, it is referred to as cutaneous melanoma. Melanoma can also occur in the eyes and is called ocular or intraocular melanoma. Melanoma occurs rarely in the meninges, the digestive tract, lymph nodes or other areas where melanocytes are found. In some embodiments, the melanoma is a metastatic melanoma. 40-60% of melanomas carry an activating mutation in the gene encoding the serine-threonine protein kinase B-RAF (BRAF). Among the BRAF mutations observed in melanoma, over 90% are at codon 600, and among these, over 90% are a single nucleotide mutation resulting in substitution of valine for glutamic acid (BRAFV600E).

As used herein, the term “treatment” or “treat” refers to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “immunotherapy” has its general meaning in the art and refers to the treatment that consists in administering an immunogenic agent i.e. an agent capable of inducing, enhancing, suppressing or otherwise modifying an immune response.

In some embodiments, the immunotherapy consists in administering the patient with at least one immune checkpoint inhibitor.

As used herein, the term “immune checkpoint inhibitor” has its general meaning in the art and refers to any compound inhibiting the function of an immune inhibitory checkpoint protein.

As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW 243.55. S70 is an anti-PD-L1 described in WO 2010/077634 A1. MEDI 4736 is an anti-PD-L 1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO 2006/121168. Merck 3745, also known as MK-3475 or SCH-900475, is an anti-PD-1 antibody described in U.S. Pat. No. 8,345,509 and WO 2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505(2004 ) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

In some embodiments, the immunotherapy consists in administering to the patient a combination of a CTLA-4 antagonist and a PD-1 antagonist.

Other immune-checkpoint inhibitors include lymphocyte activation gene-3 (LAG-3) inhibitors, such as IMP 321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18 ) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9(Gal9). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

As used herein, the term “immunotherapy resistance” refers to an acquired resistance of a cancer to the immune response induced by the immunotherapy. Therefore, a resistant tumor or tumor cell is more likely to escape and survive humoral and/or cellular immune defense mechanisms in a subject receiving the immunotherapy. The phrase “overcoming immunotherapy resistance” in context of the invention shall be effective if compared to a non-treated control, the tumor or tumor cell becomes more sensitive to an immune response induced by immunotherapy. In particular, the patient become a responder. As used herein the term “responder” in the context of the present disclosure refers to a patient that will achieve a response, i.e. a patient where the cancer is eradicated, reduced or improved after immunotherapy. According to the invention, the responders have an objective response and therefore the term does not encompass patients having a stabilized cancer such that the disease is not progressing after immunotherapy. A “non-responder” or “refractory patient” includes patients for whom the cancer does not show reduction or improvement after immunotherapy.

The term “non responder” also includes patients having a stabilized cancer. Typically, the characterization of the patient as a responder or non-responder can be performed by reference to a standard or a training set. The standard may be the profile of a patient who is known to be a responder or non-responder or alternatively may be a numerical value. Such predetermined standards may be provided in any suitable form, such as a printed list or diagram, computer software program, or other media. When it is concluded that the patient is a non-responder, the physician could take the decision to administer the agent that reduces or prevents the increase in ceramide levels. More particularly, the method of the present invention is particularly suitable for preventing tumor escape in a patient treated with immunotherapy. As used herein, the term “tumor escape” refers to any mechanism by which tumors escape the host's immune system.

In some embodiments, the immunotherapy resistance (or tumor escape) is induced by TNF-alpha. More particularly, the immunotherapy resistance results from the dedifferentiation of the tumor cells that is induced by TNF-alpha.

As used herein, the term “dedifferentiation”” refer to processes by which a tumor cell become less specialized in phenotype and broader in lineage potential.

As used herein, the term “TNFα” or “TNF-alpha” denotes the tumor necrosis factor-alpha. The human TNF-alpha is a human cytokine encoded by the TNF-alpha gene.

In some embodiments, the method of the present invention comprises administering to the patient a therapeutically effective combination of the agent that reduces or prevents the increase in ceramide levels with a TNFα blocking agent.

As used herein, the term “TNFα blocking agent” or “TBA”, it is herein meant a biological agent which is capable of neutralizing the effects of TNFα. Said agent is a preferentially a protein such as a soluble TNFa receptor, e.g. Pegsunercept, or an antibody. In some embodiments, the TBA is a monoclonal antibody having specificity for TNFα or for TNFα receptor. In some embodiments, the TBA is selected in the group consisting of Etanercept (Enbrel®), Infliximab (Remicade®), Adalimumab (Humira®), Certolizumab pegol (Cimzia®), and golimumab (Simponi®). Recombinant TNF-receptor based proteins have also been developed (e.g. etanercept, a recombinant fusion protein consisting of two extracellular parts of soluble TNFα receptor 2 (p75) joined by the Fc fragment of a human IgG1 molecule). A pegylated soluble TNF type 1 receptor can also be used as a TNF blocking agent. Additionally, thalidomide has been demonstrated to be a potent inhibitor of TNF production. TNFα blocking agents thus further include phosphodiesterase 4 (IV) inhibitor thalidomide analogues and other phosphodiesterase IV inhibitors. As used herein, the term “etanercept” or “ETA” denotes the tumor necrosis factor-alpha (TNFα) antagonist used for the treatment of rheumatoid arthritis. The term “etanercept” (ETA, ETN, Enbrel) is a recombinant TNF-receptor IgG-Fc-fusion protein composed of the p75 TNF receptor genetically fused to the Fc domain of IgG1. Etanercept neutralizes the proinflammatory cytokine tumor necrosis factor-α (TNFα) and lymphotoxin-α (Batycka-Baran et al., 2012).

As used herein, the term “ceramide” has its general meaning in the art and refers to a sphingolipid signaling molecule generated from de novo synthesis which is coordinated by serine palmitosyltransferase (SPT) and ceramide synthase (CerS), and/or from enzymatic hydrolysis of sphingomyelin coordinated by sphingomyelinases (SMases). The steady-state availability of ceramide is also regulated by ceramidases that convert ceramide to sphingosine by catalyzing hydrolysis of the ceramide amide group. One form of acid ceramidase may also be a secreted enzyme, while a form of neutral ceramidase may be mitochondrial and hence might affect ceramide synthase-mediated ceramide signaling in the mitochondria. Ceramide is also generated by enzymatic hydrolysis of sphingomyelin by sphingomyelinases. Sphingomyelin is generated by the enzyme sphingomyelin synthase (SMS) and localizes to the outer leaflet of the plasma membrane, providing a semipermeable barrier to the extracellular environment. Several isoforms of sphingomyelinase can be distinguished by pH optima for their activity, and are referred to as acid (ASMase), neutral (NSMase) or alkaline SMase. Of these isoforms, NSMase and ASMase, may be activated rapidly by diverse stressors and cause increased ceramide levels within minutes to hours. Thus ceramide levels may be reduced by the administration of any agent or agents that directly or indirectly inhibit the synthesis of ceramide or ceramide metabolic enzymes. Agents that inhibit the enzymes of both the de novo and sphingomyelinase pathways are preferred.

As used herein, the term “agent that reduces or prevents the increase in ceramide and glycosylated ceramide levels” refers to any naturally occurring or synthetically produced organic or inorganic element or composition that when administered to a subject results in a reduction of ceramide or glycosylated ceramide in the subject. A therapeutic reduction expressed as a decrease in ceramide compared to levels in the absence of ceramide synthesis inhibitor, may be between 0.001 % to 10%, 10%-20%, 20%-30%, 30%-40%, 40%-50%, 50%-60%, 70%-80%, 80%-90%, or 90%-100%, preferably greater that 10% and most preferably greater then 50% of control values. Ceramide levels may be determined through any number of techniques known to those skilled in the art including but not limited to thin layer chromatography, high-pressure liquid chromatography, mass spectrometry, immunochemical based assays and enzyme based assays, including those using ceramide kinase or diacylglycerol kinase as described by Bektas et al. (Analytical Biochemistry 320 (2003) 259-265), and Modrak (Methods in Molecular Medicine, vol. 111: Vol 2: In Vivo Models, Imaging and Molecular Regulators., Ed. Blumenthal. Humana Press Inc., NJ), and herby incorporated by reference. In particular, the agent of the present invention decreases the level of glycosphingolipids.

The agents typically include any chemical compound, or bioactive molecule derived from any living organism, including agents derived from animal, plant, fungus or bacteria, including but not limited to amino acids, polypeptides, carbohydrates, oligonucleotides, or combinations thereof, which directly or indirectly inhibit ceramide synthesis, or the synthesis of ceramide metabolic enzymes. The most well-known examples are inhibitors which target the enzymes of the de novo synthesis and sphingomyelinase pathways. For review, see Delgado et al., (Delgado et al. (2006) Biochim Biophys Acta. 1758(12):1957-77) hereby incorporated by reference and discussed below. In some embodiments, the ceramide de novo pathway compromises a series of enzymes leading to ceramide from the starting components serine and palmitoyl CoA.

In some embodiments, the agent that inhibits ceramide synthesis is selected from the group consisting of serine palmitoyltransferase inhibitors, ceramide synthase inhibitors, dihydroceramide desaturase inhibitors, sphingomyelinase inhibitors, acid sphingomyelinase inhibitors, neutral sphingomyelinase inhibitors, alkaline sphingomyelinase inhibitors physiological sphingomyelinase inhibitors, sphingomyelin analogs, scyphostatin, scyphostatin analogs, and L-carnitine.

In some embodiments, the agent that inhibits ceramide synthesis is selected from the group consisting of sphingofungins, lipoxamycin, myriocin, L-cyclosehne, β-chloro-L-alanine, Viridiofungins, Fumonisin B, Fumonisin B1, N-acylated Fumonisin B1, O-deacylated Fumonisin B1, Fumonisins, AAL-toxin, Australifungins, cyclopropene-containing sphingolipid, a-ketoamide, urea analogs of cyclopropene-containing sphingolipid, thiourea analogs of cyclopropene-containing sphingolipid, tyclodecan-9-xanthogenate, L-α-phosphatidyl-D-myo-inositol-3,5-bisphosphate, L-α-phosphatidyl-D-myo-inositol-3,4,5-thphosphate ceramide-1-phosphate, sphingosine-1-phosphate, glutathione, desipramine, imipramine, SR33557, (3-carbazol-9-yl-propyl)-[2-(3,4-dimethoxy-phenyl)-ethyl)-methyl-amine, Hexanoic acid (2-cyclo-pent-1-enyl-2-hydroxy-1-hydroxy-methyl-ethyl)-amide, C11AG, GW4869, scyphostatin, macquarinnicin A, alutenusin, chlorogentisylquinone manunnycin A, α-Mangostin, spiroepoxide 1, sphingolactones, 3-O-methylsphingomyelin, 3-O-ethylsphingomyelin, analogs of sphingolyelin, [3 (10,11-Dihydro-dibenzo [b, f] azepin-5-yl) N-propyl]-[2 (3,4-dimethoxyphenyl)-ethyl]methylamin, [3 (10,11-Dihydro-dibenzo [b, f] azepin-5-yl)-N-propyl]-[2 (4-methoxyphenyl)-ethyl] methylamin, [2 (3,4-Dimethoxyphenyl) ethyl]-[3 (2-chlorphenothiazin-IO-yl)-N-propyl]-methylamin, [2 (4-Methoxyphenyl) ethyl]-[3 (2-chlorphenothiazin-I O-yl)-N-propyl]-methylamin, [3 (Carbazol-9-yl)-N-propyl]-[2 (3,4-dimethoxyphenyl)-ethyl] methylamin, [3 (Carbazol-9-yl) N-propyl]-[2 (4-methoxyphenyl)-ethyl] methylamin, [2 (3,4-Dimethoxyphenyl)-ethyl] [2 (phenothiazin-10-yl)-N-ethyl]-methylamin, [2 (4-Methoxyphenyl)-ethyl]-[2 (phenothiazin-10-yl)-N-ethyl]-methylamin, [(3,4-Dimethoxyphenyl)-acetyl]-[3 (2-chlorphenothiazin-I O-yl)-N-propyl]-methylamin, n (1-naphthyl)-N′ [2 (3,4-dimethoxyphenyl)-ethyl]-ethyl diamine, n (1-naphthyl)-N'[2 (4-methoxyphenyl)-ethyl]-ethyl diamine, n [2 (3,4-Dimethoxyphenyl)-ethyl]-n [1-naphthylmethyl] amine, n [2 (4-Methoxyphenyl)-ethyl]-n [1-naphthylmethyl] amine, [3 (10.11-Dihydro dibenzo [b, f] azepin-5-yl)-N-propyl]-[(4-methoxyphenyl)-acetyl]-methylamin, [2 (10,11-Dihydro-dibenzo [b, f] azepin-5-yl)-N-ethyl]-[2 (3,4-dimethoxyphenyl)-ethyl] methylamin, [2 (10,11-Dihydro-dibenzo [b, f] azepin-5-yl)-N-ethyl]-[2 (4-methoxyphenyl)-ethyl]-methylamin, [2 (10,11-Dihydro-dibenzo [b, f] azepin-5-yl)-N-ethyl]-[(4-methoxyphenyl)-acetyl]-methylamin, n [2 (Carbazol-9-yl)-N-ethyl]-N′ [2 (4-methoxyphenyl)-ethyl] piperazin, 1 [2 (Carbazol-9-yl)-N-ethyl]-4 [2 (4-methoxyphenyl)-ethyl]-3,5-dimethylpiperazin, [2 (4-Methoxyphenyl)-ethyl]-[3 (phenoxazin-10-yl)-N-propyl]-methylamin, [3 (5,6,11 ,12-Tetrahydrodibenzo [b, f] azocin)-N-propyl]-[3 (4-methoxyphenyl)-propyl] methylamin, n (5H-Dibenzo [A, D] cycloheptan-5-y1)-N′ [2 (4-methoxyphenyl)-ethyl]-propylene diamine, [2 (Carbazol-9-yl)-N-ethyl]-[2 (4-methoxyphenyl)-ethyl] methylamine, scyphostatin, analogs of scyphostatin, L-carnitine, silymarin, 1-phenyl-2-decanoylaminon-3-morpholino-1-propanol, 1-phenyl-2-hexadecanoylaminon-3-pyrrolidino-1-propanol, L-camitine, human milk bile salt-stimulated lipase, myriocin, cycloserine, 1-phenyl-2-palmitoyl-3-morpholino-1-propanol, methylthiodihydroceramide, propanolol, and resveratrol, N-[(1R,2R)-1-(2,3-dihydro-1,4-benzodioxin-6-yl)-1-hydroxy-3-pyrrolidin-1-ylpropan-2-yl]octanamide; dihydro-1,4-benzodioxin-6-yl)-1-hydroxy-3-pyrrolidin-1-ylpropan-2-yl]octanamide;(2R,3R)-2,3-dihydroxybutanedioic acid. In some embodiments, the agent that reduces or prevents the increase in ceramide and glycosylated ceramide levels is not N,N′-Bis[4-(4,5-dihydro-1H-imidazol-2-yl)phenyl]-3,3′-p-phenylene-bis-acrylamide dihydrochloride (also known as GW4869).

In some embodiments, the agent of the present invention is a ceramide synthase inhibitor, in particular a glucosylceramide synthase inhibitor such as N-[(1R,2R)-1-(2,3-dihydro-1,4-benzodioxin-6-yl)-1-hydroxy-3-pyrrolidin-1-ylpropan-2-yl]octanamide;(2R,3R)-2,3-dihydroxybutanedioic acid, (2R,3R,4R,5S)-1-Butyl-2-(hydroxymethyl)-3,4,5-piperidinetriol, or (3S)-1-Azabicyclo[2.2.2]oct-3-yl {2-[2-(4-fluorophenyl)-1,3-thiazol-4-yl]-2-propanyl}carbamate.

The inhibitors of ceramide synthesis disclosed herein are non-exhaustive. One of ordinary skill in the art would appreciate that derivatives, analogs (i.e. compound having a structure similar to that of another compound but differing from certain components such as, as example, one atom, a functional group or substructures) or fragments of these inhibitors would similarly be inhibitory. In addition to the agents described herein are agents that decrease ceramide pathway metabolic enzymes, or increase ceramide catabolic enzymes, including but not limited to agents, which modify, or regulate transcriptional or translational activity or which otherwise degrade, inactivate, or protect theses enzymes.

As used herein, the term “therapeutically effective amount” is meant a sufficient amount of the agent that reduces or prevents the increase in ceramide levels for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the agent that reduces or prevents the increase in ceramide levels is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

A further object of the present invention relates to a method of determining whether a patient suffering from a cancer will achieve a response with immunotherapy comprising determining the level of at least one ceramide metabolite in a blood sample from the patient wherein said level correlates with the response of the patient to immunotherapy.

In some embodiments, the blood sample is a plasma sample.

In some embodiments, the level of at least 8 ceramide derivatives is determined in the blood sample obtained from the patient. In particular, the 8 ceramide derivatives are total trihexosylceramides, C24:0 dihexosylceramide, C20:0 trihexosylceramide, C18:0 monohexosylceramide, C16:0 dihydrosphingomyelin, C24:1 GM3 ganglioside, C14:0 dihydrosphingomyelin and C22:0 trihexosylceramide.

Typically, low levels of the ceramide derivative indicate that the patient achieves a response and conversely high levels of the ceramide derivative indicate that the patient does not achieve a response.

As used herein, the term “low” refers to a measure that is less than normal, less than a standard such as a predetermined reference value or a subgroup measure that is relatively less than another subgroup measure. For example, low ceramide derivative means a measure of the ceramide derivative that is less than a normal the ceramide derivative measure in a particular set of patient samples. A normal the ceramide derivative measure may be determined according to any method available to one skilled in the art. Low ceramide derivative may also mean a measure that is less than a predetermined reference value, such as a predetermined cutoff value. Low ceramide derivative may also mean a measure wherein a low ceramide derivative subgroup is relatively lower than another subgroup. For example, without limitation, according to the present specification, two distinct patient subgroups can be created by dividing samples around a mathematically determined point, such as, without limitation, a median, thus creating a group whose measure is low (i.e., less than the median) with respect to another group whose measure is high (i.e., greater than the median).

As used herein, the term “high” refers to a measure that is greater than normal, greater than a standard such as a predetermined reference value or a subgroup measure or that is relatively greater than another subgroup measure. For example, high ceramide derivative refers to a measure of the ceramide derivative that is greater than a normal the ceramide derivative measure. A normal ceramide derivative measure may be determined according to any method available to one skilled in the art. High ceramide derivative may also refer to a measure that is equal to or greater than a predetermined reference value, such as a predetermined cutoff. High ceramide derivative may also refer to a measure of the ceramide derivative wherein a high ceramide derivative subgroup has relatively greater levels of the ceramide derivative than another subgroup. For example, without limitation, according to the present specification, two distinct patient subgroups can be created by dividing samples around a mathematically determined point, such as, without limitation, a median, thus creating a subgroup whose measure is high (i.e., higher than the median) and another subgroup whose measure is low. In some cases, a “high” level may comprise a range of level that is very high and a range of level that is “moderately high” where moderately high is a level that is greater than normal, but less than “very high”.

In some embodiments, the predetermined reference value is a threshold value or a cut-off value that can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the the ceramide derivative level in the sample, one can use algorithmic analysis for the statistic treatment of the the ceramide derivative level determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the method comprises the steps of i) determining the level of at least one ceramide metabolite in the blood sample, ii) comparing the level determined at step i) with a predetermined reference level wherein differential between the determined level and the predetermined reference level indicates whether the patient will achieve or not a response to immunotherapy.

In some embodiments, when level of the ceramide derivative is higher that its predetermined reference level, it is concluded that the patient will not achieve a response to immunotherapy (i.e. “non responder”).

In some embodiments, when the level of the ceramide derivative is lower that its predetermined reference level, it is concluded that the patient will achieve a response.

In some embodiments, the predetermined reference level is the level of the ceramide derived determined before the administration of the immunotherapy (“baseline value”).

In some embodiments, the level of the ceramide derivative is determined along the therapy, wherein an increase of the level of ceramide derivative indicates that the patient will not achieve a response, and conversely a decrease of the level of the ceramide derivative indicates that the patient will achieve a response.

The invention will be further illustrated by the following figures and examples. However, these examples and figures should not be interpreted in any way as limiting the scope of the present invention.

### FIGURES

FIG. 1. TNF modulates the expression of genes involved in ceramide metabolism regulation. Gene Ontology pathway analysis from RNA Seq data generated in the human melanoma cell line WM 35 incubated with 50 ng/mL TNF for 24 h as compared to untreated cells (n=4).

FIG. 2: TNF increases the levels of various ceramide metabolites. The human melanoma cell line (WM 35) was incubated with or without 50 ng/ml TNF for 48 h. Intracellular ceramide metabolites were quantified by mass spectrometry. Ceramide metabolites, which significantly increased upon TNF treatment, are indicated (SM: sphingomyelin; dhSM: dihydrosphingomyelin; Cer: ceramide; dhCer: dihydroceramide; HexCer: monhexosylceramide; CDH: dihexosylceramide). Numbers of carbons and double bonds on the fatty acid linked to the sphingosine backbone are indicated, respectively.

FIG. 3: Exogenous ceramides trigger melanoma cell dedifferentiation. The WM35 melanoma cell line was incubated with (C2-Cer) or without (NT) cell permeant ceramides (ceramides C2:0) for 24 h. Melan-a expression was evaluated by flow cytometry. (MFI: Median Fluorescent Intensity).

FIG. 4: Fumonisin B1 impairs TNF-induced melanoma cell dedifferentiation. The WM 35 melanoma cell line was incubated with 5 μM fumonisin B1 (FB1) and 50 ng/ml TNF for 72 hours. Melan-a expression was evaluated by flow cytometry.

FIG. 5: Targeting the glucosylceramide synthase impairs TNF-induced tyrosinase expression inhibition in the B16Ova mouse melanoma cell line. B16Ova cells were incubated in the presence or absence of 50 ng/ml TNF and 10 μM eliglustat for 24 (A), 48 (B) or 72 (C) hours. RT-qPCR was performed to assess tyrosinase mRNA expression. Data are means±sem (n=6).

FIG. 6. Plasma ceramide metabolites predict resistance to immunotherapy in advanced melanoma patients. Ceramide metabolite plasma levels were measured in advanced melanoma patients treated with ipilimumab (anti-CTLA-4) and nivolumab (anti-PD-1) in combination (patients from TICMEL clinical trial) or not (patients from MELANFa clinical trial) with anti-TNF at baseline and week 6 post-treatment induction (n=48 paired pre/post treatment including 25 responders and 23 progressors). A, Sensitivity and specificity of this score to predict the clinical response was determined. B, Comparison of ceramide metabolite evolution between patients from TICIMEL, treated with tritherapy (ipilimumab+nivolumab+certolizumab or infliximab) (n=27), and MELANF alpha treated with bitherapy (ipilimumab+nivolumab) (n=21) clinical trials.

### EXAMPLE

Incubation of melanoma cell lines with recombinant TNF triggered a melanoma cell dedifferentiation process as evaluated by transcriptomic, western blot and flow cytometry experiments (data not shown).

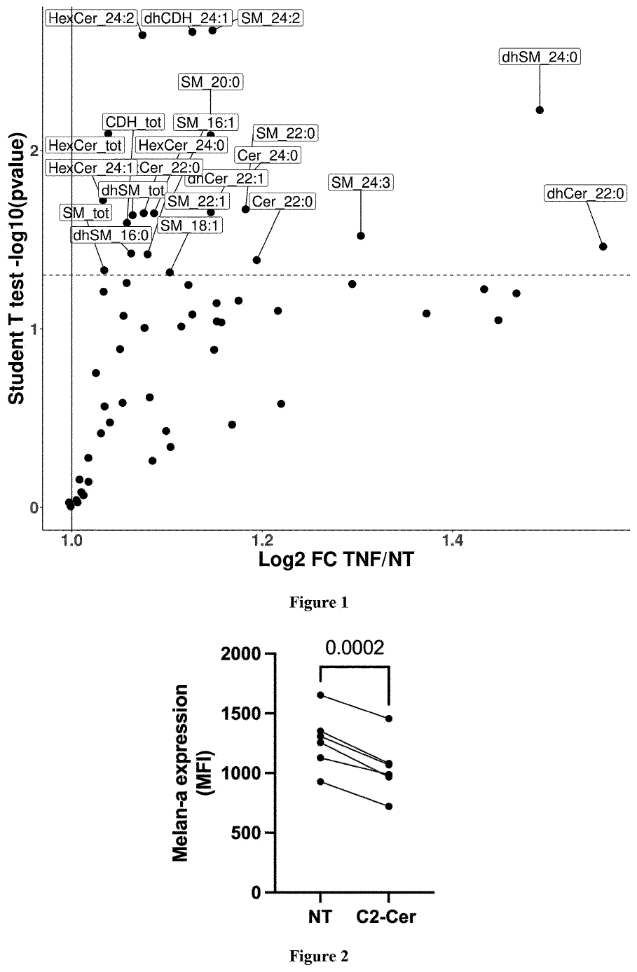

Analysis of the gene ontology sphingolipid pathways from the RNA Seq experiments suggested that TNF likely increased the sphingolipid biosynthetic process while reducing the ganglioside biosynthetic process via lactosylceramide (i.e., dihexosylceramide) (FIG. 1).

TNF triggered the increase of various ceramide derivatives as evaluated by mass spectrometry, including some (dihydro)sphingomyelins, (dihydro)ceramides, monohexosylceramides and dihexosylceramides (FIG. 2).

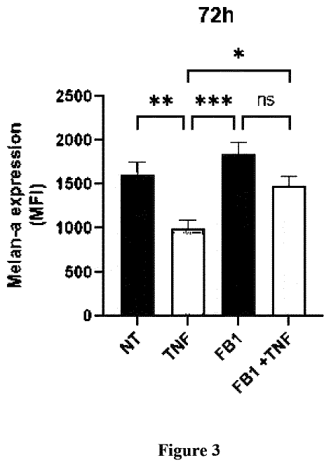

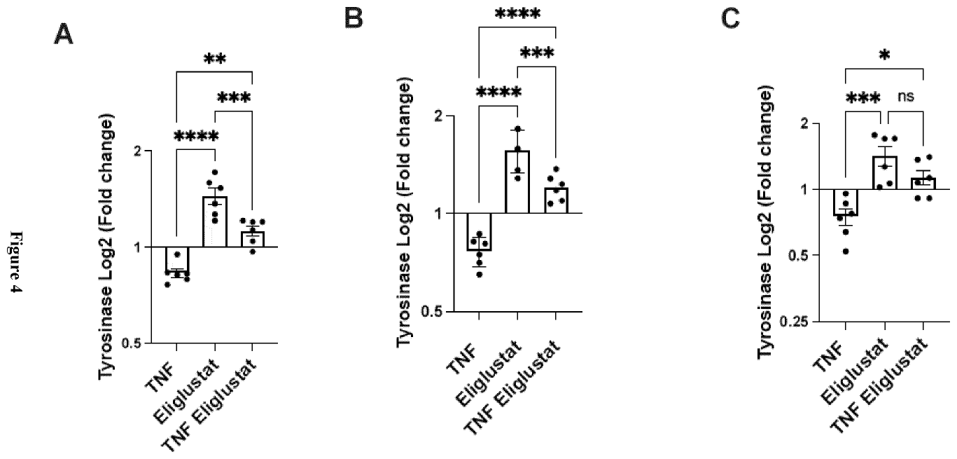

Incubation of melanoma cells with exogenous ceramides triggered the decrease of Melan-A expression, indicating that ceramides or ceramide derivatives can induce melanoma cell dedifferentiation (FIG. 3).

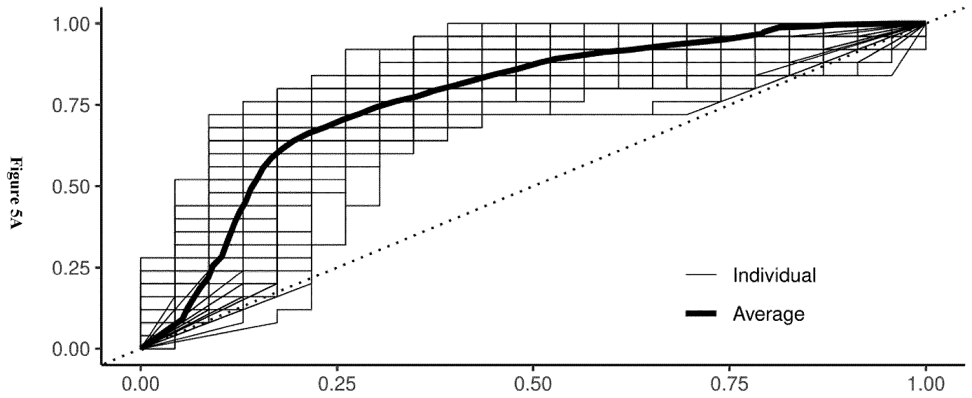

TNF-induced melanoma cell dedifferentiation was impaired by fumonisin B1, an inhibitor of the de novo ceramide synthesis, further indicating that ceramides or ceramide derivatives can induce melanoma cell dedifferentiation upon TNF signaling (FIG. 4).

TNF-induced melanoma cell dedifferentiation was associated with an upregulation of the expression of the UGCG gene, which encodes the glucosylceramide synthase (Data not shown). To evaluate the contribution of the glycosphingolipid pathway in the TNF-induced melanoma cell dedifferentiation process, we monitored the impact of eliglustat, an inhibitor of glucosylceramide synthase (GCS), by RT-qPCR on various markers of melanoma dedifferentiation. As expected, TNF decreased the gene expression of MITF and its targets MLNA, DCT and TYR, and, at the opposite, increased the expression of the stemness factor NGFR in 451Lu melanoma cells (data not shown). Combining TNF with the inhibitor of GCS (eliglustat) partially hindered its impact on these changes (data not shown). This was confirmed at the protein level where combining TNF with eliglustat partially reversed TNF-induced decrease of Melan-A (data not shown) and tended to reverse TNF-induced NGFR expression (data not shown).

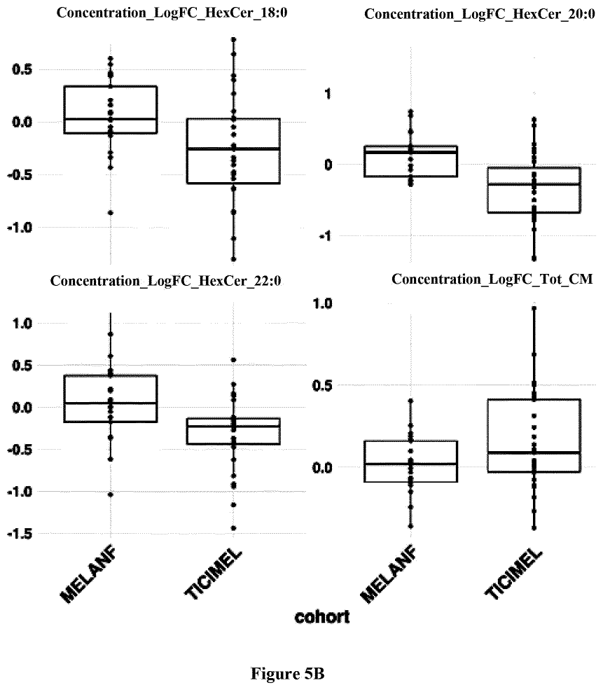

The impact of GCS inhibition on TNF-induced melanoma dedifferentiation was further evaluated in the mouse B 16Ova melanoma cell line in which TNF incubation for 24 to 72 hours triggered the decrease of Tyrosinase expression as evaluated by RT-qPCR (FIG. 5). Eliglustat significantly attenuated this phenomenon in response to TNF (FIG. 5).

Eliglustat significantly increased Tyrosinase expression levels as evaluated by simple western compared to B16Ova melanoma cells incubated in the presence or absence of TNF (data not shown).

We previously showed that TNF contributes to limit anti-PD-1 efficacy in mouse cancer models, including melanoma, and that administering anti-TNF antibodies enhance the efficacy of anti-PD-1 therapy in mice. Our preclinical work constitutes the scientific rationale of two clinical trials MELANFa (NCT03348891) and TICIMEL (NCT03293784) in advanced melanoma patients. We took advantage of those clinical trials in advanced melanoma patients treated with ipilimumab+nivolumab alone (MELANFα) or in combination with anti-TNF (TICIMEL) (paired pre and post treatment samples, n=48). To evaluate whether plasma levels of ceramide metabolites can predict the clinical outcome in patients at week 12, ceramide metabolites in plasma were quantified by mass spectrometry at baseline and at week 6 post-treatment induction. Then, we used the Boruta algorithm to determine the sphingolipids whose evolution along immunotherapy can predict the clinical response. Among 78 sphingolipids, the top 8 sphingolipids the increase of which predict the clinical response were mainly glycosphingolipids, including the total amount of trihexosylceramides (CTH), C24:0 dihexosylceramide, C20:0 trihexosylceramide, C18:0 monohexosylceramide, C16:0 dihydroshingomyelin, C24:1 GM3 ganglioside, C14:0 dihydrosphingomyelin and C22:0 trihexosylceramide (data not shown). Using these top 8 sphingolipids, we created a sphingolipid score predictive of the response to ICI. Indeed, in our cohorts of patients, a low sphingolipid score was significantly associated with a better response to ICI (data not shown) and predicted as much as 77% of the response (FIG. 6A).

To evaluate the impact of TNF-dependent signaling on the evolution of the plasma sphingolipidome between baseline and week 6, we compared the sphingolipid pattern in the plasma of advanced melanoma patients treated with ipilimumab and nivolumab (i.e., bi-therapy) from the MELANFα clinical trial, with those measured in the TICIMEL clinical trial (NCT03293784), in which patients were co-administered with ipilimumab, nivolumab and anti-TNF (certolizumab or infliximab) (i.e., tri-therapy). When co-administered with ipilimumab and nivolumab, anti-TNF decreased the monohexosylceramide plasma content, with significant differences for C18:0, C20:0 and C22:0 monohexosylceramides, and increased the total sphingomyelin plasma content (FIG. 6B).

Collectively, our data indicate that the glycosphingolipid pattern in plasma may predict the clinical outcome of advanced melanoma patients treated with ipilimumab and nivolumab. Moreover, the TNF-dependent signaling pathway contributes to produce circulating monohexosylceramides, which are precursors for more complex glycosphingolipids putatively involved in resistance to ICI.

### REFERENCES

Throughout this application, various references describe the state of the art to which this invention pertains. The disclosures of these references are hereby incorporated by reference into the present disclosure.
Wolchok J D, Chiarion-Sileni V, Gonzalez R, Grob J J, Rutkowski P, Lao C D, Cowey C L, Schadendorf D, Wagstaff J, Dummer R, Ferrucci P F, Smylie M, Butler M O, Hill A, Márquez-Rodas I, Haanen JBAG, Guidoboni M, Maio M, Schöffski P, Carlino M S, Lebbé C, McArthur G, Ascierto P A, Daniels G A, Long G V, Bas T, Ritchings C, Larkin J, Hodi F S. Long-Term Outcomes With Nivolumab Plus Ipilimumab or Nivolumab Alone Versus Ipilimumab in Patients With Advanced Melanoma. J Clin Oncol. 2022 Jan. 10;40(2):127-137.
Montfort A, Bertrand F, Rochotte J, Gilhodes J, Filleron T, Milhes J, Dufau C, Imbert C, Riond J, Tosolini M, Clarke C J, Dufour F, Constantinescu A A, Junior N F, Garcia V, Record M, Cordelier P, Brousset P, Rochaix P, Silvente-Poirot S, Therville N, Andrieu-Abadie N, Levade T, Hannun Y A, Benoist H, Meyer N, Micheau O, Colacios C, Ségui B. Neutral Sphingomyelinase 2 Heightens Anti-Melanoma Immune Responses and Anti-PD-1 Therapy Efficacy. Cancer Immunol Res. 2021 May;9(5):568-582.
Imbert C, Montfort A, Fraisse M, Marcheteau E, Gilhodes J, Martin E, Bertrand F, Marcellin M, Burlet-Schiltz O, Peredo A G, Garcia V, Carpentier S, Tartare-Deckert S, Brousset P, Rochaix P, Puisset F, Filleron T, Meyer N, Lamant L, Levade T, Ségui B, Andrieu-Abadie N, Colacios C. Resistance of melanoma to immune checkpoint inhibitors is overcome by targeting the sphingosine kinase-1. Nat Commun. 2020 Jan. 23;11(1):437.
Carrié L, Virazels M, Dufau C, Montfort A, Levade T, Ségui B, Andrieu-Abadie N. New Insights into the Role of Sphingolipid Metabolism in Melanoma. Cells. 2020 Aug. 26;9(9):1967. Bilal F, Montfort A, Gilhodes J, Garcia V, Riond J, Carpentier S, Filleron T, Colacios C, Levade T, Daher A, Meyer N, Andrieu-Abadie N, Ségui B. Sphingomyelin Synthase 1 (SMS1) Downregulation Is Associated With Sphingolipid Reprogramming and a Worse Prognosis in Melanoma. Front Pharmacol. 2019 Apr. 30;10:443. doi: 10.3389/fphar.2019.00443.
Levade T, Andrieu-Abadie N, Micheau O, Legembre P, Ségui B. Sphingolipids modulate the epithelial-mesenchymal transition in cancer. Cell Death Discov. 2015 Oct. 12;1:15001.
Montfort A, Colacios C, Levade T, Andrieu-Abadie N, Meyer N, Ségui B. The TNF Paradox in Cancer Progression and Immunotherapy. Front Immunol. 2019 Jul. 31;10:1818.
Bertrand F, Montfort A, Marcheteau E, Imbert C, Gilhodes J, Filleron T, Rochaix P, Andrieu-Abadie N, Levade T, Meyer N, Colacios C, Ségui B. TNFα blockade overcomes resistance to anti-PD-1 in experimental melanoma. Nat Commun. 2017 Dec. 22;8(1):2256.
Perez-Ruiz E, Minute L, Otano I, Alvarez M, Ochoa M C, Belsue V, de Andrea C, Rodriguez-Ruiz M E, Perez-Gracia J L, Marquez-Rodas I, Llacer C, Alvarez M, de Luque V, Molina C, Teijeira A, Berraondo P, Melero I. Prophylactic TNF blockade uncouples efficacy and toxicity in dual CTLA-4 and PD-1 immunotherapy. Nature. 2019 May;569(7756):428-432.
Landsberg J, Kohlmeyer J, Renn M, Bald T, Rogava M, Cron M, Fatho M, Lennerz V, Wölfel T, Hölzel M, Tüting T. Melanomas resist T-cell therapy through inflammation-induced reversible dedifferentiation. Nature. 2012 Oct. 18;490(7420):412-6.

1. A method of overcoming immunotherapy resistance in a patient suffering from cancer comprising administering to the patient a therapeutically effective amount of an agent that reduces or prevents an increase in ceramide and glycosylated ceramide levels.

2. The method of claim 1 wherein the patient suffers from melanoma.

3. The method of claim 1 wherein the immunotherapy comprises at least one immune checkpoint inhibitor.

4. The method of claim 3 wherein the immune checkpoint inhibitor is selected from the group consisting of PD-1 antagonists, PD-L1 antagonists, PD-L2 antagonists, CTLA-4 antagonists, VISTA antagonists, TIM-3 antagonists, LAG-3 antagonists, IDO antagonists, KIR2D antagonists, A2AR antagonists, B7-H3 antagonists, B7-H4 antagonists, and BTLA antagonists.

5. The method of claim 3 wherein the immunotherapy comprises administering a combination of a CTLA-4 antagonist and a PD-1 antagonist.

6. The method of claim 1 wherein the agent that inhibits ceramide synthesis is selected from the group consisting of serine palmitoyltransferase inhibitors, ceramide synthase inhibitors, dihydroceramide desaturase inhibitors, sphingomyelinase inhibitors, acid sphingomyelinase inhibitors, neutral sphingomyelinase inhibitors, alkaline sphingomyelinase inhibitors physiological sphingomyelinase inhibitors, sphingomyelin analogs, scyphostatin, scyphostatin analogs, and L-carnitine.

7. The method of claim 6 wherein the agent that inhibits ceramide synthesis is selected from the group consisting of sphingofungins, lipoxamycin, myriocin, L-cyclosehne, β-chloro-L-alanine, Viridiofungins, Fumonisin B, Fumonisin B1, N-acylated Fumonisin B1, O-deacylated Fumonisin B1, Fumonisins, AAL-toxin, Australifungins, cyclopropene-containing sphingolipid, α-ketoamide, urea analogs of cyclopropene-containing sphingolipid, thiourea analogs of cyclopropene-containing sphingolipid, tyclodecan-9-xanthogenate, L-α-phosphatidyl-D-myo-inositol-3,5-bisphosphate, L-α-phosphatidyl-D-myo-inositol-3,4,5-thphosphate ceramide-1-phosphate, sphingosine-1-phosphate, glutathione, desipramine, imipramine, SR33557, (3-carbazol-9-yl-propyl)-[2-(3,4-dimethoxy-phenyl)-ethyl)-methyl-amine, Hexanoic acid (2-cyclo-pent-1-eny1-2-hydroxy-1-hydroxy-methyl-ethyl)-amide, C11AG, GW4869, scyphostatin, macquarinnicin A, alutenusin, chlorogentisylquinone manunnycin A, α-Mangostin, spiroepoxide 1, sphingolactones, 3-O-methylsphingomyelin, 3-O-ethylsphingomyelin, analogs of sphingolyelin, [3 (10,11-Dihydro-dibenzo [b, f] azepin-5-yl)-N-propyl]-[2 (3,4-dimethoxyphenyl)-ethyl] methylamin, [3 (10,11-Dihydro-dibenzo [b, f] azepin-5-yl)-N-propyl]-[2 (4-methoxyphenyl)-ethyl] methylamin, [2 (3,4-Dimethoxyphenyl)-ethyl]-[3 (2-chlorphenothiazin-IO-yl)-N-propyl]-methylamin, [2 (4-Methoxyphenyl)-ethyl]-[3 (2-chlorphenothiazin-I O-yl)-N-propyl]-methylamin, [3 (Carbazol-9-yl)-N-propyl]-[2 (3,4-dimethoxyphenyl)-ethyl] methylamin, [3 (Carbazol-9-yl)-N-propyl]-[2 (4-methoxyphenyl)-ethyl] methylamin, [2 (3,4-Dimethoxyphenyl)-ethyl]-[2 (phenothiazin-10-yl)-N-ethyl]-methylamin, [2 (4-Methoxyphenyl)-ethyl]-[2 (phenothiazin-10-yl)-N-ethyl]-methylamin, [(3,4-Dimethoxyphenyl)-acetyl]-[3 (2-chlorphenothiazin-I O-yl)-N-propyl]-methylamin, n (1-naphthyl)-N′ [2 (3,4-dimethoxyphenyl)-ethyl]-ethyl diamine, n (1-naphthyl)-N'[2 (4-methoxyphenyl)-ethyl]-ethy] diamine, n [2 (3,4-Dimethoxyphenyl)-ethyl]-n [1-naphthylmethyl] amine, n [2 (4-Methoxyphenyl)-ethyl]-n [1-naphthylmethyl] amine, [3 (10.11-Dihydro dibenzo [b, f] azepin-5-yl)-N-propyl]-[(4-methoxyphenyl)-acetyl]-methylamine, [2 (10,11-Dihydro-dibenzo [b, f] azepin-5-yl)-N-ethyl]-[2 (3,4-dimethoxyphenyl)-ethyl] methylamine, [2 (10,11-Dihydro-dibenzo [b, f] azepin-5-yl)-N-ethyl]-[2 (4-methoxyphenyl)-ethyl]-methylamine, [2 (10,11-Dihydro-dibenzo [b, f] azepin-5-yl)-N-ethyl]-[(4-methoxyphenyl)-acetyl]-methylamine, n [2 (Carbazol-9-yl)-N-ethyl]-N′ [2 (4-methoxyphenyl)-ethyl] piperazine, 1 [2 (Carbazol-9-yl)-N-ethyl]-4 [2 (4-methoxyphenyl)-ethyl]-3,5-dimethylpiperazine, [2 (4-Methoxyphenyl)-ethyl]-[3 (phenoxazin-10-yl)-N-propyl]-methylamine, [3 (5,6,11,12-Tetrahydrodibenzo [b, f] azocin)-N-propyl]-[3 (4-methoxyphenyl)-propyl] methylamine, n (5H-Dibenzo [A, D] cycloheptan-5-yl)-N′ [2 (4-methoxyphenyl)-ethyl]-propylene diamine, [2 (Carbazol-9-yl)-N-ethyl]-[2 (4-methoxyphenyl)-ethyl] methylamine, scyphostatin, analogs of scyphostatin, L-carnitine, silymarin, 1-phenyl-2-decanoylamino-3-morpholino-1-propanol, 1-phenyl-2-hexadecanoylamino-3-pyrrolidino-1-propanol, L-camitine, myriocin, cycloserine, 1-phenyl-2-palmitoyl-3-morpholino-1-propanol, methylthiodihydroceramide, propranolol, resveratrol, N-[(1R,2R)-1-(2,3-dihydro-1,4-benzodioxin-6-yl)-1-hydroxy-3-pyrrolidin-1-ylpropan-2-yl]octanamide; and (2R,3R)-2,3-dihydroxybutanedioic acid.

8. The method of claim 6 wherein, the agent is a ceramide synthase inhibitor.

9. The method of claim 1 further comprising administering to the patient a TNFα blocking agent.

10. A method of determining whether a patient suffering from a cancer will achieve a response with immunotherapy and treating the patient comprising
i) measuring a level of at least one ceramide metabolite in a blood sample from the patient,
ii) determining that the level is lower than a predetermined reference value; and
iii) treating the patient determined to have a low level of at least one ceramide metabolite with immunotherapy.

11. The method of claim 10 wherein the level of at least 8 ceramide metabolites is determined in the blood sample obtained from the patient.

12. The method of claim 10 wherein a low levels of the at least one ceramide metabolite indicates that the patient will achieve a response to the immunotherapy and a high level of the at least one ceramide metabolite indicates that the patient will not achieve a response to the immunotherapy.

13. (canceled)

14. (canceled)

15. The method of claim 10, wherein the predetermined reference level is a level of the at least one ceramide metabolite determined before administration of the immunotherapy.

16. The method according to claim 10, wherein the level of the at least one ceramide metabolite is determined during the immunotherapy, and wherein an increase of the level of the at least one ceramide metabolite indicates that the patient is not achieving a response, and a decrease of the level of the at least one ceramide metabolite indicates that the patient is achieving a response.

17. The method of claim 8, wherein the agent is a ceramide synthase inhibitor.

18. The method of claim 17, wherein the ceramide synthase inhibitor is selected from the group consisting of N-[(1R,2R)-1-(2,3-dihydro-1,4-benzodioxin-6-yl)-1-hydroxy-3-pyrrolidin-1-ylpropan-2-yl]octanamide; (2R,3R)-2,3-dihydroxybutanedioic acid, (2R,3R,4R,5S)-1-butyl-2-(hydroxymethyl)-3,4,5-piperidinetriol, and (3S)-1-azabicyclo[2.2.2]oct-3-yl{2-[2-(4-fluorophenyl)-1,3-thiazol-4-yl]-2-propanyl} carbamate.

19. The method of claim 11, wherein the at least 8 ceramide derivatives are total trihexosylceramides, C24:0 dihexosylceramide, C20:0 trihexosylceramide, C18:0 monohexosylceramide, C16:0 dihydrosphingomyelin, C24:1 GM3 ganglioside, C14:0 dihydrosphingomyelin and C22:0 trihexosylceramide.
